(19) United States
(12) Patent Application Publication
(10) Pub. No.: US 2024/0118204 A1
(43) Pub. Date: Apr. 11, 2024

(54) Fluorescence Turn-On Detection of Nitric Oxide Free Radical Using a Simple Semiconducting Nanocrystal (QD) and Ferric Dithiocarbamate Complex

(71) Applicant: The Government of the United States of America, as represented by the Secretary of the Navy, Arlington, VA (US)

(72) Inventors: Okhil K. Nag, Brandywine, MD (US); Michael H. Stewart, Springfield, VA (US); Bethany Almeida; James Delehanty, Washington, DC (US)

(21) Appl. No.: 18/374,796

(22) Filed: Sep. 29, 2023

Related U.S. Application Data
(60) Provisional application No. 63/413,390, filed on Oct. 5, 2022.

(51) Int. Cl. G01N 21/64; C09K 11/02; C09K 11/88; G01N 33/00

(52) U.S. Cl. CPC G01N 21/6428; C09K 11/025; C09K 11/883; G01N 33/0037

(57) ABSTRACT

A semiconducting nanocrystal (quantum dot or QD) and ferric dithiocarbamate complex (QD-Fe(DTC)₃) operate as a sensing system to detect nitric oxide (NO) in ambient conditions using fluorescence resonance energy transfer (FRET). The sensing system comprises two components: (1) an energy donor in the form of a dihydrolipoic acid (DHLA)-coated QD with strong fluorescence emission, and (2) an energy acceptor in the form of a ferric ion-dithiocarbamate complex (Fe(DTC)₃) that binds onto the QD surface via carboxylate coordination. The ferric ion in the QD-Fe(DTC)₃ complex acts a strong energy acceptor, resulting in weak fluorescence (FL) emission (“turn-off”) of the QD when excited using 405 nm light. In the presence of NO, ferric ion (3⁺) reduces to ferrous (2⁺), with decreased ability to accept energy from the QD, in turn appearing as increased FL emission from the QD (“turn-on”) as a sensing signal.

### CROSS-REFERENCE TO RELATED APPLICATIONS

This application claims the benefit of U.S. Provisional Application 63/413,390 filed on Oct. 5, 2022, the entirety of which is incorporated herein by reference.

### FEDERALLY-SPONSORED RESEARCH AND DEVELOPMENT

The United States Government has ownership rights in this invention. Licensing inquiries may be directed to Office of Technology Transfer, US Naval Research Laboratory, Code 1004, Washington, DC 20375, USA; +1.202.767.7230; techtran@nrl.navy.mil, referencing NC 210976.

### BACKGROUND

The small molecule free radical nitric oxide (NO) plays numerous critical roles in mammalian cellular physiology and homeostasis. Since its discovery, various investigations have established NO as an essential signaling molecule in multiple physiological systems. It also plays a role in protecting the body from attack by foreign pathogens such as viruses and bacteria. In the environment, NO free radical is one of the most critical primary pollutants directly emitted from industrial combustion processes. Direct inhalation of NO can harm the respiratory tract. Also, ambient NO is converted into nitrogen dioxide (NO₂), responsible for acid rain and environmental damage. Detection of NO within living cells or environmental samples remains a considerable challenge due to NO's short half-life, complicated instrumental setup for its measurement, and multistep preparation of sensing reagents. Fluorescence-based detection of NO (using molecular fluorophores) has remained the most popular method of choice to date due to the minimal instrumental setup required for the measurement. Organic fluorescence dyes, such as diaminonaphthalene, and its derivatives, are specific and sensitive to NO. However, such organic fluorescence probes generally exhibit narrow excitation bands and broad emission spectra with a tendency to photobleach quickly, and they are used at high concentrations within cells, which is often toxic. A need exists for a simple NO detection scheme with good signal-to-noise characteristics.

### BRIEF SUMMARY

A semiconducting nanocrystal (a quantum dot or QD) and ferric dithiocarbamate complex (QD-Fe(DTC)₃) operate as a sensing system to detect nitric oxide (NO) in ambient conditions using fluorescence resonance energy transfer (FRET). The sensing system comprises two components: (1) an energy donor in the form of a dihydrolipoic acid (DHLA)-coated QD with strong fluorescence emission, and (2) an energy acceptor in the form of a ferric ion-dithiocarbamate complex (Fe(DTC)₃) that binds onto the QD surface via carboxylate coordination. The ferric ion in the QD-Fe(DTC)₃ complex acts a strong energy acceptor, resulting in weak fluorescence (FL) emission (“turn-off”) of the QD when excited using 405 nm light. In the presence of NO, ferric ion (3+) reduces to ferrous (2+), with decreased ability to accept energy from the QD, in turn appearing as increased FL emission from the QD (“turn-on”) as a sensing signal.

In one embodiment, a sensor for nitric oxide comprises a CdSe/ZnS quantum dot (QD) coated with dihydrolipoic acid; and a ferric ion-dithiocarbamate complex (Fe(DTC)₃) bound to the QD via carboxylate coordination to a ZnS surface thereof, wherein the QD and Fe(DTC)₃ are configured as donor and acceptor, respectively, in fluorescence resonance energy transfer.

In another embodiment, a method of preparing a sensor for nitric oxide, the method comprises providing a dihydrolipoic acid-coated quantum dot (QD); and contacting the QD with ferric dithiocarbamate so that it binds thereto via carboxylate coordination, thereby obtaining a QD-Fe(DTC)₃ pair, where the QD and Fe(DTC)₃ are configured as donor and acceptor, respectively, in fluorescence resonance energy transfer.

In a further embodiment, a method of sensing nitric oxide (NO) comprises providing a sensor comprising a quantum dot (QD) coated with dihydrolipoic acid and a ferric ion-dithiocarbamate complex (Fe(DTC)₃) bound thereto via carboxylate coordination wherein the QD and Fe(DTC)₃ are configured as donor and acceptor, respectively, in fluorescence resonance energy transfer (FRET); contacting the sensor with NO, thereby causing a change in FRET emission from the sensor; and measuring the change in FRET emission, wherein the change therein reflects NO in contact with the sensor.

### BRIEF DESCRIPTION OF THE DRAWINGS

FIG. 1 depicts the proposed QD-based FRET sensing of nitric oxide (NO) in an aqueous solution. The left side shows the general structures of DHLA-encapsulated 525 nm and 625 nm emitting QDs with strong fluorescence emission of 525 nm emitting QD are shown at the bottom. The top center of the figure provides a schematic representation of QDs (525 nm and 625 nm) with bound Fe(DTC)₃. The larger 625 nm QDs enable complexation with a higher number of Fe(DTC)3 due to their larger surface area compared to the smaller 525 nm QDs. Bound Fe(DTC)₃ on the QD surface acts as a strong energy acceptor via FRET, resulting in quenched QD emission (“QD off”). As seen in the right side of the figure, nitric oxide (NO) binds with Fe(DTC)₃ associated with the QD surface and reduces its ability to accept energy from QD, resulting in the recovery of QD emission (“QD on”). The bottom right portion of the figure shows the resulting increased optical emission.

FIGS. 2A-2D show steady state quenching of QD fluorescent emission (FL) by Fe(DTC)₃ is dependent on (Fe(DTC)₃/QD ratio and QD size. FIGS. 2A and 2B show the quenching spectra of DHLA-capped QD 525 nm (FIG. 2A) and QD 625 nm (FIG. 2B) upon addition of increasing concentrations Fe(DTC)₃. FIG. 2C shows the normalized quenching efficiency of Fe(DTC)₃ as a function of Fe(DTC)₃/QD ratio derived from the plots FIG. 2A and FIG. 2B for QD 525 nm and QD 625 nm, respectively. The arrows indicate the ratio where 50% FL quenching of 525 nm and 625 nm QDs was achieved. FIG. 2D depicts the proposed binding mechanism of Fe(DTC)₃ on the QD surface. Fe(DTC)₃ binds with the ZnS shell on the QD via carboxyl coordination.

FIGS. 3A-3F show results of nitric oxide (NO) sensing using QD-Fe(DTC)₃ complex. FIGS. 3A-3D show the FL emission spectra of QD-Fe(DTC)₃ for 625 nm (FIGS. 3A and 3B) and 525 nm (FIGS. 3C and 3D) QDs upon addition of two different concentration of diethylamine NONOate diethylammonium salt (NONOate) NO donor; 25 μM (FIGS. 3A and 3C) and 2.5 μM (FIGS. 3B and 3D). Spectra were taken every 1 min after initial addition of NONOate, until (up to 7 min) no change in emission was observed. FIGS. 3E and 3F show normalized percent increase in FL emission upon addition of 25 μM (FIG. 3E) and 2.5 μM (FIG. 3F) of NONOate with time.

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

### Overview

As described herein, semiconducting nanocrystals (QDs) serve as a fluorescence platform to sense NO in an aqueous solution. QDs have been shown to have superior photophysical properties, including (1) strong fluorescence (FL) emission that is resistant to photobleaching, (2) broad absorption, (3) narrow and size-dependent FL spectra, and, most importantly, (4) their ability to act as an excellent energy donor in Forster resonance energy transfer (FRET) configurations.

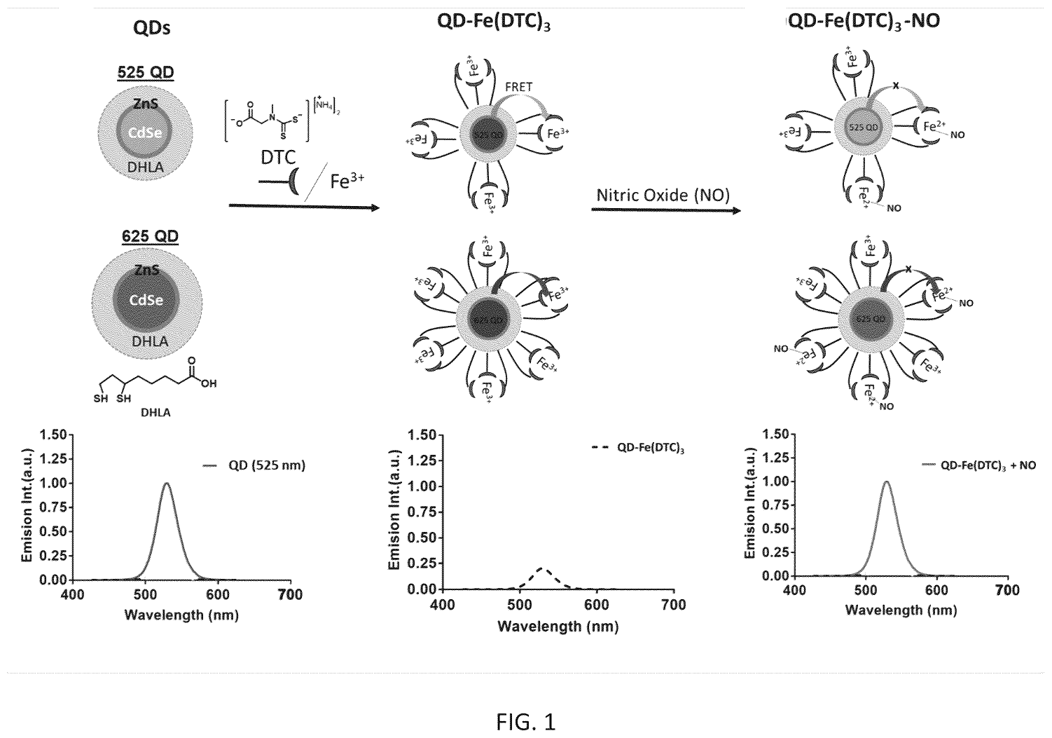

The FRET efficiency in the QD-Fe(DTC)₃ system pair was theoretically calculated as a function of varying ratios of Fe(DTC)₃ arrayed around the QD. This was done for two different sized QDs: 525 nm emitting (with diameter 4.1 nm) and 625 nm emitting (9.4 nm diameter) (Figure 1i). From our FRET analysis, the overlap integrals between Fe(DTC)₃ and the QDs are 1.54×10⁻¹⁴ cm³M⁻¹ and 1.45×10⁻¹⁴ cm³M⁻¹, for the 525 nm and 625 nm QDs, respectively. While the overlap integrals are similar, the FRET efficiency (FRET_{E}) between a single donor and acceptor is inversely proportional to r⁶, where r is the separation distance between the donor and acceptor. Thus, the smaller 525 nm QD donor is expected to engage in more efficient FRET than the larger 625 nm QD. Since our system involves a single QD donor interacting with multiple acceptors arrayed concentrically around the QD surface, FRET_{E} also increases with increasing number of acceptors (n). Based on this, we anticipated that a larger size 625 QD would need to bind with more Fe(DTC)₃ to induce similar quenching as smaller 525 QD, due to more available surface area (Figure 1ii). This larger amount of Fe(DTC)₃ on the larger size 625 QD would then be expected to bind with more NO, providing a stronger NO sensing signal and a larger dynamic range for NO sensing (Figure 1iii).

### Examples

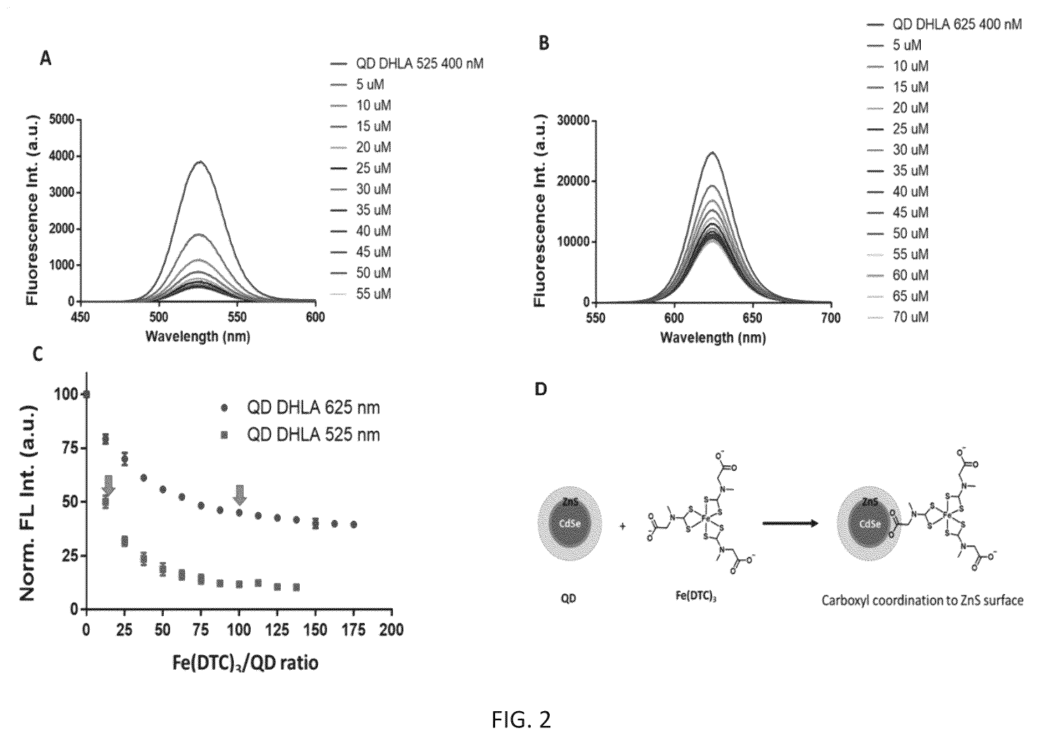

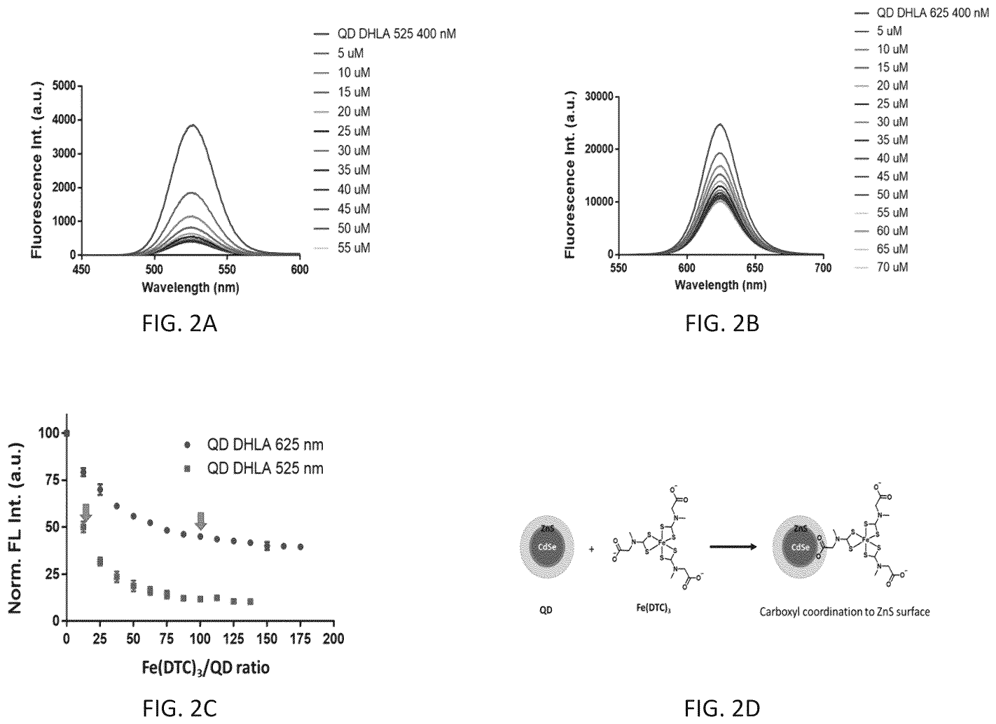

The sensing scheme used CdSe/ZnS core/shell QDs coated with DHLA ligands, prepared as described in Stewart et al. and Nag et al., as donor scaffolds for the FRET-based sensors. The QDs were assembled with ferric dithiocarbamate (Fe(DTC)₃) so that the carboxyl groups in the Fe(DTC)₃ coordinated to the ZnS shell of the QDs (FIG. 2D). Briefly, Fe(DTC)₃ was freshly and rapidly generated from FeCl₃·6H₂O and [DTC][NH₄]₂ under nitrogen in Tris buffer (0.05M, pH 7.5) at an Fe-to-DTC ratio of 1:3. QDs were self-assembled with the Fe(DTC)₃ at desired ratios in Tris buffer (0.05M, pH 7.5) for five minutes. The results was the QD-Fe(DTC)₃ FRET pairs, where the QD and Fe(DTC)₃ act as energy donor and acceptor, respectively.

The steady-state FL spectra of the QDs (400 nM) was measured with an increasing ratio of Fe(DTC)₃ to assay the degree of quenching due to energy transfer from the QD to Fe(DTC)₃. As shown in FIGS. 2A & B, adding each bolus (5 μM) of Fe(DTC)3 species induces increasing FL quenching efficiency for both QDs. After reaching a certain QD:Fe(DTC)3 ratio, the FL quenching remains constant, suggesting saturation of the Fe(DTC)3 complexation on the surface of the QD. In these systems, ˜50% FL quenching was achieved at ratios of 12 and 100 species Fe(DTC)₃ for the 525 nm QDs and 625 nm QDs, respectively (arrows in FIG. 2C). Additionally, at their saturation, a maximum of about 85% and 55% quenching efficiency were observed for 525 nm QD and 625 nm QDs, with Fe(DTC)3/QD ratio 80 and 125, respectively. At saturation, the FRET E approaches 88% (525 nm QD) and 57% (625 nm QD). These results are believed to relate to the closer center to center distance (r) between the smaller 525 nm QD and the Fe(DTC)₃ compared with the larger 625 nm QD. Indeed, the average separation distance, calculated from FRET data, was smaller for the 525 nm QD (4.4 nm) than the 625 nm QD (6.2 nm).

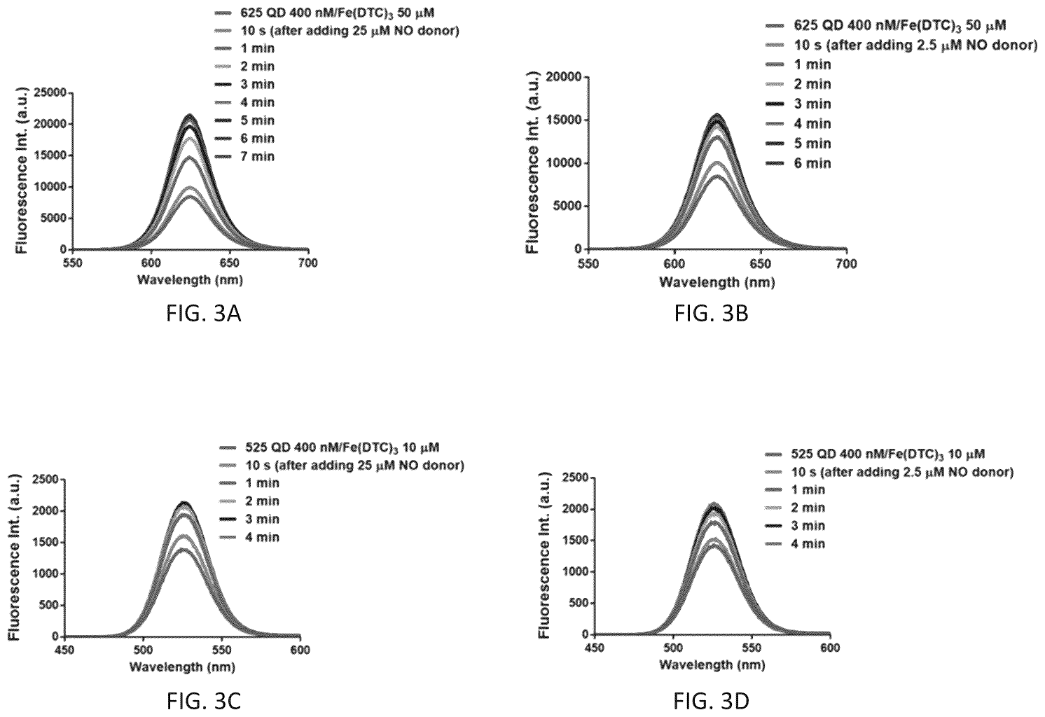

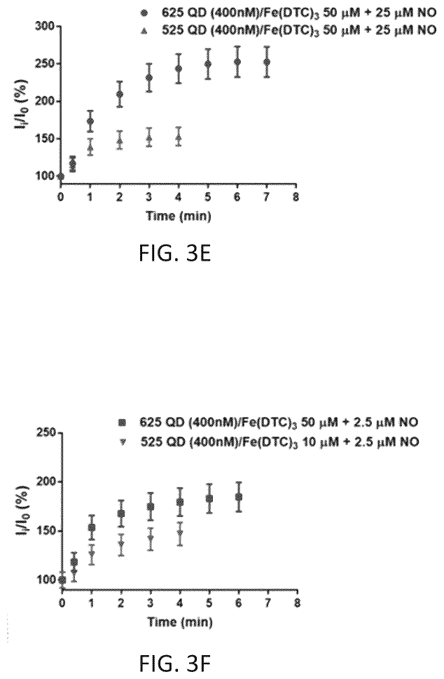

The NO sensing capabilities of Fe(DTC)₃/QD systems with 525 nm QD and 625 nm QD with two different concentrations (25 μM in FIGS. 3A and 3C and 2.5 μM in FIGS. 3B and 3D) of a NONOate NO donor added to the surrounding solution. As shown in FIGS. 3A-3D, with the addition of NONOate, FL emission of Fe(DTC)₃/QD starts to increase (“QD on”) immediately, which progress with time as the NONOate releases more NO into the solution. After a certain time point (4-7 min) Fe(DTC)₃/QD FL emission observed no significant changes for both concentrations (25 and 2.5 μM) NONOate, suggesting no significant release of the NO from the added NONOate after that time. To quantify the NO sensing signal, we plotted Iᵢ/I₀ vs. time, where I₀ is the FL emission intensities of Fe(DTC)₃/QD before adding NONOate (t=0), and Iᵢ is the FL emission intensities of Fe(DTC)₃/QD after adding NONOate. After 1 min of 25 μM NONOate addition, the Fe(DTC)₃/QD (525 nm) and Fe(DTC)₃/QD (625 nm) systems showed ˜140% and ˜-174% change in the FL emission signal, suggesting efficient and fast NO sensing capabilities of these systems. Additionally, the maximum signal for Fe(DTC)₃/QD (525 nm) and Fe(DTC)₃/QD (625 nm) was measured to be about 150% and 250% before plateauing at 2 min and 5 min, respectively. These results demonstrate that the larger Fe(DTC)₃/QD (625 nm) system is more sensitive and offers a wider dynamic range for sensing NO oxide compared to the smaller Fe(DTC)₃/QD (525 nm). A similar trend was observed when Fe(DTC)₃/QD (525 nm) and Fe(DTC)₃/QD (625 nm) were tested with a lower concentration (2.5 μM) of NONOate.

### Further Embodiments

Potential uses of the invention include the commercial sale and use of the materials in application areas where NO sensing is needed in ambient conditions without a need for complicated electrochemical instruments. Such applications include the determination of NO production and extracellular release in cultured cells, tissue slices, and environmental sample, such as pollutants from industrial combustion processes. Other applications include the determination of the rate of NO release for organic molecules by simple fluorescence measurement in ambient conditions.

QD emission can be measured using techniques and equipment known in the art. By way of non-limiting example, this can include spectroscopy, multi-well plates, and combinations thereof.

### Advantages

The reported QD-Fe(DTC)₃ complex (QD-Fe(DTC)₃) efficiently detects nitric oxide (NO) in solution in ambient conditions. The formation strategy for this QDs and Fe(DTC)₃ is straightforward, fast and can be assembled immediately prior to the nitric oxide sensing experiment.

This technique offers several advantages over the prior art. First, it does not require QD encapsulation with polymers, which involves multiple chemical synthesis steps. Second, because the DHLA ligand (˜1.1.nm in length) system used here is significantly shorter in length compared to bulky polymers used in the prior art, the separation distance between the QD donor and the Fe acceptor is minimized, resulting in more efficient FRET and NO sensing. Third, the nature of the assembly allows for fine control over the number of Fe(DTC)₃ acceptors arrayed around the central QD. Finally, controlling the size of the central QD donor/scaffold offers the ability to control the dynamic range and sensitivity of NO sensing.

### CONCLUDING REMARKS

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

a) Synthesis of DHLA QD
Stewart, M. H., et al., Competition between Förster resonance energy transfer and electron transfer in stoichiometrically assembled semiconductor quantum dot-fullerene conjugates. ACS Nano, 2013. 7(10): 9489-9505.
Okhil K. N., et al., Quantum Dot-Peptide-Fullerene Bioconjugates for Visualization of in Vitro and in Vivo Cellular Membrane Potential. ACS Nano 2017, 11, 6, 5598-5613

b) Synthesis of Dithiocarbamate and QD-Fe(DTC)₃ and NONOate Nitric Oxide Donor
Wang, Suhua, et al., Nitric Oxide Switches on the Photoluminescence of Molecularly Engineered Quantum Dots, J. Am. Chem Soc. 2009, 131, 11692-11694.
Jian S., et al., Fluorescence Turn-On Detection of Gaseous Nitric Oxide Using Ferric Dithiocarbamate Complex Functionalized Quantum Dots. Anal. Chem. 2014, 86, 5628-5632.

What is claimed is:

1. A sensor for nitric oxide comprising:
a CdSe/ZnS core/shell quantum dot (QD) coated with dihydrolipoic acid; and
a ferric ion-dithiocarbamate complex (Fe(DTC)₃) bound to the QD via carboxylate coordination to a ZnS surface thereof, wherein the QD and Fe(DTC)₃ are configured as donor and acceptor, respectively, in fluorescence resonance energy transfer.

2. A method of preparing a sensor for nitric oxide, the method comprising:
providing a dihydrolipoic acid-coated CdSe/ZnS core/shell quantum dot (QD); and
contacting the QD with ferric dithiocarbamate so that it binds thereto via carboxylate coordination to a ZnS surface thereof,
thereby obtaining a QD-Fe(DTC)₃ pair, where the QD and Fe(DTC)₃ are configured as donor and acceptor, respectively, in fluorescence resonance energy transfer.

3. A method of sensing nitric oxide (NO), the method comprising:
providing a sensor comprising a CdSe/ZnS core/shell quantum dot (QD) coated with dihydrolipoic acid and a ferric ion-dithiocarbamate complex (Fe(DTC)₃) bound thereto via carboxylate coordination to a ZnS surface of the QD,
wherein the QD and Fe(DTC)₃ are configured as donor and acceptor, respectively, in fluorescence resonance energy transfer (FRET);
contacting the sensor with NO, thereby causing a change in FRET emission from the sensor; and
measuring the change in FRET emission, wherein the change therein reflects NO in contact with the sensor.
